(19) United States
(12) Patent Application Publication
(10) Pub. No.: US 2026/0283480 A1
(43) Pub. Date: Sep. 24, 2026

(54) VITAL SIGN MONITORING SYSTEM FOR DETECTING MULTIPLE VITAL SIGNS OF MULTIPLE PERSONS IN A SCENE AND METHOD THEREOF

(71) Applicant: Delta Electronics Int'l (Singapore) Pte Ltd, Singapore (SG)

(72) Inventors: Yuan Liu, Singapore (SG); Xuemei Fu, Singapore (SG); Xingze Wang, Singapore (SG); Andy Khong, Singapore (SG); Tzu-Yi Hung, Singapore (SG)

(21) Appl. No.: 19/354,628

(22) Filed: Oct. 9, 2025

(30) Foreign Application Priority Data
Mar. 24, 2025 (SG) 10202500763Y
Aug. 28, 2025 (SG) 10202502453W

(51) Int. Cl. A61B 5/0205; A61B 5/00; A61B 5/021; A61B 5/024; A61B 5/05; A61B 5/08; G01S 13/88

(52) U.S. Cl. CPC A61B 5/0205; A61B 5/05; A61B 5/7257; A61B 5/726; G01S 13/88; A61B 5/021; A61B 5/02444; A61B 5/0816; A61B 2560/0223

(57) ABSTRACT

A vital sign monitoring system for detecting multiple vital signs of multiple persons in a scene. The vital sign monitoring system includes a radar transceiver, a signal processing unit, a biometric processing unit, and a vital sign estimation unit. The radar transceiver is configured to transmit radar signals toward the scene and receive reflected signals corresponding to the multiple persons. The signal processing unit is configured to process the reflected signals to obtain phase signals corresponding to the multiple persons, and to apply wavelet transformation on the phase signals using adjustable wavelet parameters to generate, for each of the multiple persons, a respiration rate signal and a heartbeat rate signal. The biometric processing unit is configured to process the heartbeat rate signal of each of the multiple persons using a biometric model to determine a blood pressure signal of the person.

### CROSS REFERENCE TO RELATED APPLICATIONS

This application claims priority of Singapore Patent Application No. 10202500763Y, filed on Mar. 24, 2025, and Singapore Patent Application No. 10202502453 W, filed on Aug. 28, 2025, the entirety of which is incorporated by reference herein.

### TECHNICAL FIELD

The present disclosure relates to a vital sign monitoring system, and, in particular, to a vital sign monitoring system for detecting multiple vital signs of multiple persons in a scene.

### BACKGROUND

Current vital sign detection technologies have been widely applied in various fields such as health monitoring, smart healthcare, home care, and disaster rescue. Among these applications, non-contact methods for measuring vital signs, such as respiratory rate, heart rate, and blood pressure, have gained significant attention due to their low invasiveness and high convenience. However, most existing non-contact vital sign monitoring systems rely on single-point radar or optical sensors (e.g., camera imaging) to acquire physiological signals, and are primarily designed for single-person scenarios. As a result, they are ineffective in complex environments where multiple individuals are present simultaneously.

In multi-person environments, the weak physiological signals generated by different individuals, such as chest displacement, can interfere with each other. Furthermore, natural and involuntary body movements (e.g., Random Body Movements, RBM) introduce additional signal variability, which severely degrades detection accuracy. Environmental factors such as background clutter, signal reflections, and occlusions further compromise the stability and reliability of detection. Existing technologies often struggle to differentiate between multiple sources of vital sign signals, leading to inaccurate physiological parameter analysis in multi-person scenes.

In addition, signal strength and resolution tend to degrade over distance, making it difficult to maintain accurate physiological data acquisition in large or open spaces. With respect to blood pressure (BP) estimation, conventional systems often rely on indirect inference from chest displacement phase signals. However, chest movement caused by natural respirations or RBM introduces considerable clutter into the data, thereby reducing the accuracy and consistency of blood pressure estimations.

On the other hand, some systems utilize camera-based image processing methods to analyze vital signs. While these methods may offer certain levels of accuracy, they are highly dependent on lighting conditions and camera angles, and their performance may be adversely affected by skin tone variations and visual obstructions. Moreover, the use of optical imaging raises privacy concerns, particularly in public or domestic settings, thereby limiting the widespread adoption of such solutions.

Accordingly, there is an urgent need for an innovative technology capable of overcoming the above limitations.

### BRIEF SUMMARY

An embodiment of the present disclosure provides a vital sign monitoring system for detecting multiple vital signs of multiple persons in a scene. The vital sign monitoring system includes a radar transceiver, a signal processing unit, a biometric processing unit, and a vital sign estimation unit. The radar transceiver is configured to transmit radar signals toward the scene and receive reflected signals corresponding to the multiple persons. The signal processing unit is configured to process the reflected signals to obtain phase signals corresponding to the multiple persons, and to apply wavelet transformation on the phase signals using adjustable wavelet parameters to generate, for each of the multiple persons, a respiration rate signal and a heartbeat rate signal. The biometric processing unit is configured to process the heartbeat rate signal of each of the multiple persons using a biometric model to determine a blood pressure signal of the person. The vital sign estimation unit is configured to estimate, for each of the multiple persons, a respiration rate value, a heartbeat rate value, and a blood pressure value based on the respiration rate signal, the heartbeat rate signal, and the blood pressure signal of the person, respectively.

In an embodiment, the signal processing unit further includes a 2-D localization unit, a DC offset calibration unit, and a phase demodulation unit. The 2-D localization unit is configured to apply a Fast Fourier Transform to the reflected signals to obtain range and azimuth information, and apply a beamformer based on the range and azimuth information to determine enhanced signals with respect to each of the multiple persons. The DC offset calibration unit is configured to compensate for a phase shift of the enhanced signals. The phase demodulation unit is configured to derive the phase signals from the enhanced signals with the phase shift compensated.

In an embodiment, the signal processing unit includes a signal preprocessing unit and a clutter rejection unit. The signal preprocessing unit is configured to normalize the reflected signals. The clutter rejection unit is configured to eliminate environmental clutter from the normalized reflected signals. The 2-D localization unit receives the normalized reflected signals with environmental clutter eliminated from the clutter rejection unit.

In an embodiment, the signal processing unit is further configured to decompose the phase signals into resonance components having cardiopulmonary signals with the phase signals being sparse in a wavelet domain based on the wavelet transformation.

In an embodiment, the signal processing unit is further configured to determine an optimal wavelet basis based on an optimal value of the adjustable wavelet parameters that matches the cardiopulmonary signals for each of the multiple persons, and reconstructs a respiration waveform and a heartbeat waveform from the cardiopulmonary signals based on the optimal wavelet basis.

In an embodiment, the adjustable wavelet parameters include a Q factor, a redundancy factor, and a decomposition level.

In an embodiment, the Q factor evaluates resonance quality of the phase signals, the redundancy factor controls a frequency response overlap between adjacent wavelets, and the decomposition level determines a frequency span of the adjacent wavelets.

In an embodiment, the signal processing unit is further configured to determine the optimal value of the Q factor based on maximum reconstruction energy of the cardiopulmonary signals.

In an embodiment, the respiration waveform and the heartbeat waveform are reconstructed based on the optimal value of the Q factor with corresponding center frequencies falling within a respiration waveform frequency range and a heartbeat waveform frequency range, respectively.

In an embodiment, the biometric processing unit determines the blood pressure signal based on the heartbeat waveform using the biometric model, and the vital sign estimation unit estimates the respiration rate value based on the respiration waveform, and the heartbeat rate value and the blood pressure value based on the heartbeat waveform.

In an embodiment, the biometric model is a two-element Windkessel model.

An embodiment of the present disclosure further provides a method for detecting multiple vital signs of multiple persons in a scene. The method includes transmitting radar signals toward the scene and receiving reflected signals corresponding to the multiple persons by a radar transceiver, and executing steps by a processor. The executing steps include processing the reflected signals to obtain phase signals corresponding to the multiple persons, applying wavelet transformation on the phase signals using adjustable wavelet parameters to generate, for each of the multiple persons, a respiration rate signal and a heartbeat rate signal, processing the heartbeat rate signal of each of the multiple persons using a biometric model to generate a blood pressure signal of the person, and estimating, for each of the multiple persons, a respiration rate value, a heartbeat rate value, and a blood pressure value based on the respiration rate signal, the heartbeat rate signal, and the blood pressure signal of the person, respectively.

In an embodiment, the step of processing the reflected signals to obtain phase signals corresponding to the multiple persons further includes applying a Fast Fourier Transform to the reflected signals to obtain range and azimuth information, applying a beamformer based on the range and azimuth information to determine enhanced signals with respect to each of the multiple persons, compensating for a phase shift of the enhanced signals, and deriving the phase signals from the enhanced signals with the phase shift compensated.

In an embodiment, the step of processing the reflected signals to obtain phase signals corresponding to the multiple persons includes normalizing the reflected signals, and eliminating environmental clutter from the normalized reflected signals. The step of eliminating the environmental clutter is performed before the step of applying the Fast Fourier Transform to obtain the range and azimuth information.

In an embodiment, the step of processing the reflected signals to obtain phase signals corresponding to the multiple persons further includes decomposing the phase signals into resonance components having cardiopulmonary signals with the phase signals being sparse in a wavelet domain based on the wavelet transformation.

In an embodiment, the step of applying wavelet transformation on the phase signals using adjustable wavelet parameters to generate, for each of the multiple persons, a respiration rate signal and a heartbeat rate signal includes determining an optimal wavelet basis based on an optimal value of the adjustable wavelet parameters that matches the cardiopulmonary signals for each of the multiple persons, and reconstructing a respiration waveform and a heartbeat waveform from the cardiopulmonary signals based on the optimal wavelet basis.

In an embodiment, the step of applying the wavelet transformation on the phase signals using adjustable wavelet parameters to generate, for each of the multiple persons, the respiration rate signal and the heartbeat rate signal further includes determining the optimal value of the Q factor based on maximum reconstruction energy of the cardiopulmonary signals.

In an embodiment, the step of processing the heartbeat rate signal of each of the multiple persons using the biometric model to determine the blood pressure signal of the person includes determining the blood pressure value based on the heartbeat waveform using the biometric model. The step of estimating, for each of the multiple persons, the respiration rate value, the heartbeat rate value, and the blood pressure value based on the respiration rate signal, the heartbeat rate signal, and the blood pressure signal of the person includes estimating the respiration rate value and the heartbeat rate value based on the respiration waveform and the heartbeat waveform, respectively.

An embodiment of the present disclosure further provides a vital sign monitoring system for detecting multiple vital signs of multiple persons in a scene. The vital sign monitoring system includes a radar transceiver, a processor, and a storage unit. The radar transceiver is configured to transmit radar signals toward the scene and receive reflected signals corresponding to the multiple persons. The storage unit is coupled to the processor, and configured to store a computer program including instructions. When the instructions are executed by the processor, which causes the processor to process the reflected signals to obtain phase signals corresponding to the multiple persons, and to apply wavelet transformation on the phase signals using adjustable wavelet parameters to generate, for each of the multiple persons, a respiration rate signal and a heartbeat rate signal, process the heartbeat rate signal of each of the multiple persons using a biometric model to determine a blood pressure signal of the person, and estimate, for each of the multiple persons, a respiration rate value, a heartbeat rate value, and a blood pressure value based on the respiration rate signal, the heartbeat rate signal, and the blood pressure signal of the person, respectively.

In an embodiment, the phase signals are decomposed into resonance components having cardiopulmonary signals with the phase signals being sparse in a wavelet domain based on the wavelet transformation. The adjustable wavelet parameters include a Q factor, a redundancy factor, and a decomposition level. The Q factor evaluates resonance quality of the phase signals, and the redundancy factor controls a frequency response overlap between adjacent wavelets, and the decomposition level determines a frequency span of the adjacent wavelets.

The various embodiments disclosed herein provide a system capable of simultaneous multi-person detection in a shared environment, which incorporates several key technical advantages to address real-world challenges in contactless vital sign monitoring. The system exhibits strong robustness against background clutter and random body movements, enables long-range monitoring, and achieves accurate estimation of vital signs such as blood pressure, heartbeat rate, and respiration rate at the same distance. Accordingly, the proposed system significantly broadens the practical applicability, reliability, and scalability of contactless vital sign detection technologies. It is particularly well-suited for use in smart healthcare systems, remote patient monitoring, ambient assisted living, sleep health analytics, and group health and safety surveillance, among other domains. These advancements mark a substantial step forward in enabling high-fidelity, multi-user physiological sensing in unconstrained and real-world environments.

### BRIEF DESCRIPTION OF THE DRAWINGS

The present disclosure can be more fully understood by reading the subsequent detailed description and examples with references made to the accompanying drawings, wherein:

FIG. 1 is a schematic diagram of a vital sign monitoring system, according to an embodiment of the present disclosure;

FIG. 2 is a flow diagram of a method for detecting multiple vital signs of multiple persons in a scene, according to an embodiment of the present disclosure;

FIG. 3 is a schematic diagram illustrating the FMCW-based MIMO radar architecture of a radar transceiver in FIG. 1, according to an embodiment of the present disclosure;

FIG. 4 is a functional block diagram of a signal processing unit in FIG. 1, according to an embodiment of the present disclosure;

FIG. 5A presents an exemplary range profile spectrum before clutter rejection;

FIG. 5B presents an exemplary range profile spectrum after clutter rejection;

FIG. 6A is a schematic diagram illustrating the circle fitting applied to the phase data before and after DC offset calibration, according to an embodiment of the present disclosure;

FIG. 6B is a schematic diagram illustrating the extracted phase information, according to an embodiment of the present disclosure;

FIG. 7 presents an exemplary reconstructed heartbeat waveform, according to an embodiment of the present disclosure; and

FIG. 8 is a system block diagram of a vital sign monitoring system, according to an embodiment of the present disclosure.

### DETAILED DESCRIPTION

The following description is made for the purpose of illustrating the general principles of the disclosure and should not be taken in a limiting sense. The scope of the disclosure is best determined by reference to the appended claims.

In each of the following embodiments, the same reference numbers represent identical or similar elements or components.

Ordinal terms used in the claims, such as “first,” “second,” “third,” etc., are only for convenience of explanation, and do not imply any precedence relation between one another.

The descriptions provided below for embodiments of devices or systems are also applicable to embodiments of methods, and vice versa.

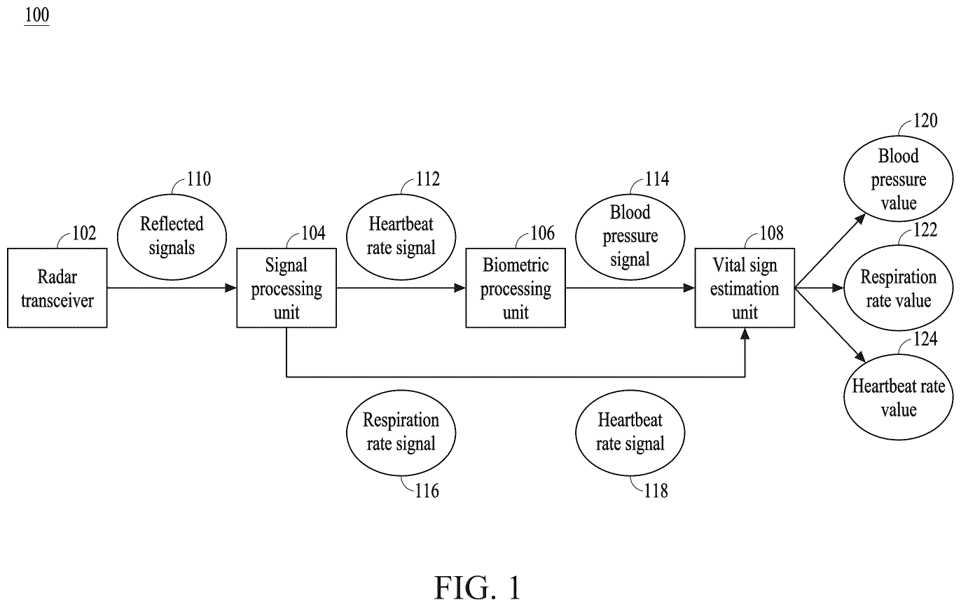

FIG. 1 is a schematic diagram of a vital sign monitoring system 100, according to an embodiment of the present disclosure. As shown in FIG. 1, the vital sign monitoring system 100 includes a radar transceiver 102, a signal processing unit 104, a biometric processing unit 106, and a vital sign estimation unit 108.

The radar transceiver 102 may include one or more radio frequency (RF) front-end modules, analog filters, frequency synthesizers, mixers, analog-to-digital converters (ADCs), and digital signal processing units configured to transmit radar signals and receive reflected signals indicative of physiological activities.

The signal processing unit 104, the biometric processing unit 106, and the vital sign estimation unit 108 may be implemented as dedicated hardware logic, such as an application-specific integrated circuit (ASIC), a field-programmable gate array (FPGA), or discrete hardware components. In some embodiments, two or more of the signal processing unit 104, the biometric processing unit 106, and the vital sign estimation unit 108 may be integrated into a single hardware module or combined into a system-on-chip (SoC) architecture to achieve improved performance, reduced size, and lower power consumption.

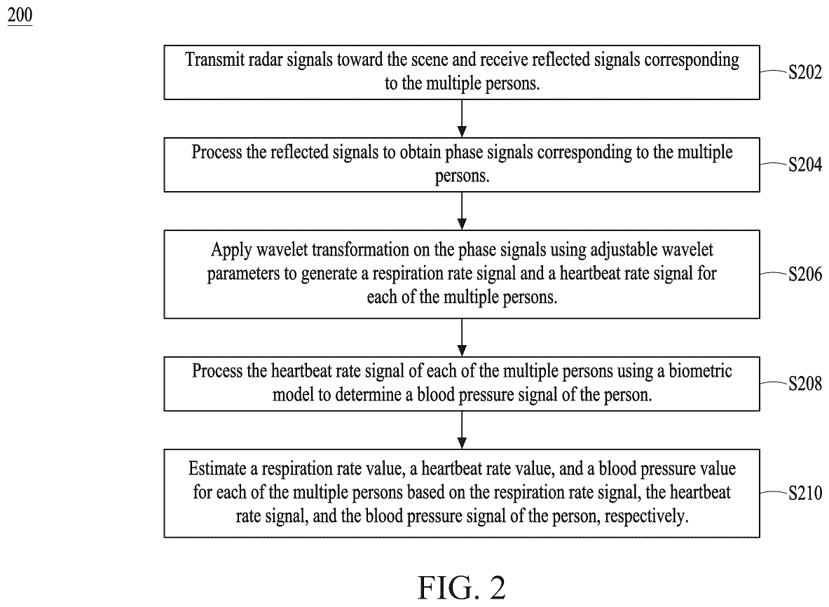

FIG. 2 is a flow diagram of a method 200 for detecting multiple vital signs of multiple persons in a scene, according to an embodiment of the present disclosure. As shown in FIG. 2, the method 200 may include steps S202-S210. These steps are performed by the components illustrated in FIG. 1. Accordingly, FIGS. 1 and 2 should be referred to collectively for a better understanding of the embodiment.

In step S202, the radar transceiver 102 transmits radar signals toward the scene and receives reflected signals corresponding to the multiple persons. The radar signals may include frequency-modulated continuous wave (FMCW) signals, pulsed radar signals, or other forms of millimeter-wave or microwave signals. The scene may include one or more persons, such as multiple users located within an indoor environment, individuals situated in a vehicle cabin, occupants present in a public transport, patients resting in a clinical care area, or potential survivors trapped under collapsed debris after an earthquake, but the present disclosure is not limited thereto.

When the transmitted radar signals impinge on the body surfaces of the persons, reflected signals are generated. The radar transceiver 102 is further configured to receive these reflected signals and convert them into digital signals for subsequent processing. The received reflected signals may originate from different individuals and may exhibit varying time delays, Doppler frequency shifts, or reflection intensities, which can be used for subsequent target detection, tracking, and classification.

In some embodiments, the radar transceiver 102 includes multiple antenna array elements to enable beamforming and spatial resolution capabilities. In particular, the radar transceiver 102 is implemented as a Multiple-Input Multiple-Output (MIMO) radar, which includes a plurality of transmit antennas and a plurality of receive antennas. The MIMO radar refers to a radar system architecture that utilizes multiple transmit (Tx) and multiple receive (Rx) antennas to form a virtual array with a significantly increased aperture. By independently controlling the signals transmitted from each Tx antenna and coherently combining the signals received by the Rx antennas, the MIMO radar can synthesize a larger number of virtual channels, thereby enhancing angular resolution, target detection capability, and spatial diversity.

In some embodiments, the MIMO radar enables spatial multiplexing and high-resolution imaging by leveraging the virtual antenna array generated from the combinations of the Tx and Rx elements. This configuration significantly improves the radar's ability to detect, separate, and track multiple targets in complex environments and allows the system to differentiate reflected signals from different individuals based on angular information, such as azimuth and elevation. The system may cooperate with a digital signal processing circuit to perform range estimation, velocity estimation, and angle-of-arrival estimation, thereby generating occupancy maps or movement trajectory information associated with the persons.

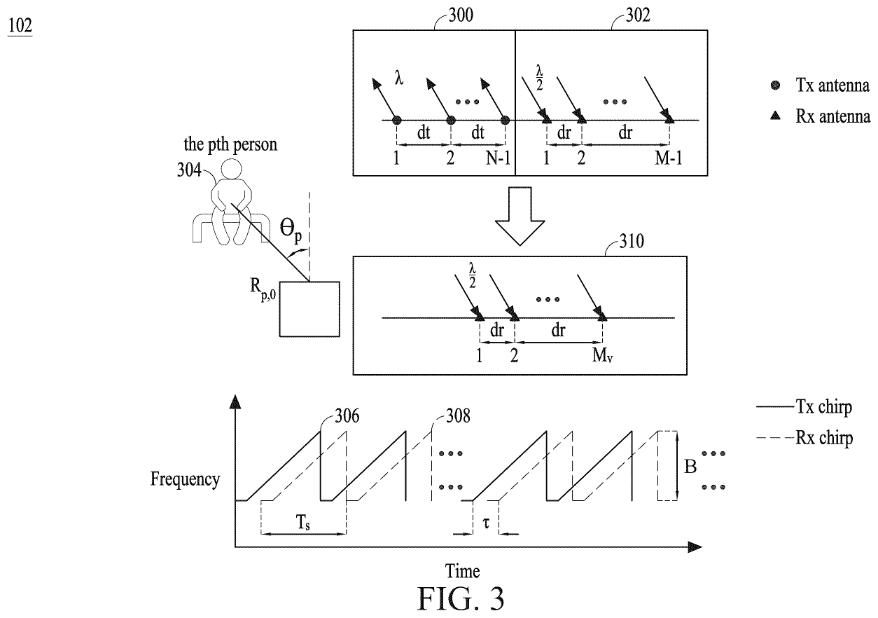

Referring to FIG. 3, FIG. 3 is a schematic diagram illustrating the FMCW-based MIMO radar architecture of a radar transceiver 102 in FIG. 1, according to an embodiment of the present disclosure. In this embodiment, the FMCW-based MIMO radar architecture is configured to perform simultaneous multi-target tracking and spatial resolution. The FMCW-based MIMO radar architecture includes a uniform linear array (ULA) of N transmit (Tx) antennas 300, spaced at an interval of dt, and a ULA of M receive (Rx) antennas 302, spaced at an interval of dr. The Tx antennas 300 are indexed as n=0, 1, . . . , N−1, and the Rx antennas 302 are indexed as m=0, 1, . . . , M−1, where N and M are positive integers.

Assuming that there are P persons within the radar's field of view, each person is indexed as p=1, . . . , P. The pth person 304 is located at a radial distance R_{p,0} from the radar origin and is oriented at an azimuth angle θₚ. The chirp signal 306 transmitted from the nth Tx antenna is defined as:

$${S}_{n} ( t ) = {A}_{t} {e}^{j 2 π} ( {f}_{0} t + B / 2 {Tt}^{2} + {φ}_{n , 0} ( t ) + {u}_{n} / λsin ( {θ}_{p} ) ) \qquad equation ( 1 )$$

where Aₜ is the amplitude of the chirp signal 306, f₀ is the starting frequency of the chirp signal 306, B is the bandwidth of the chirp signal 306, T is the duration of the chirp signal 306, φ_{n,0}(t) is the initial phase of the nth transmitter, uₙ is the position of the nth Tx antenna in the array, λ is the wavelength of the chirp signal 306, and θₚ is the direction angle of the pth person 304.

The chirp signal 306 incorporates both time-domain frequency modulation and spatial-domain directional encoding, thereby enabling joint range-angle resolution.

The chirp signals 308 received at the mth Rx antenna from the nth Tx antenna, in response to the reflection from all targets, is formulated as:

$${x}_{m , n} ( t ) = {Σ}_{p = 1}^{P} {α}_{m , n}^{( p )} s ( t - {τ}_{m , n}^{( p )} ( t ) ) + {\tilde{δ}}_{m , n} ( t ) \qquad equation ( 2 )$$

where α_{m,n}^{(p)} is the reflection coefficient associated with the pth person 304 for the (n,m) Tx-Rx antenna pair, τ_{m,n}^{(p)}(t) is the time delay corresponding to the pth person 304, and \tilde{δ}_{m,n}(t) is the clutter and noise components.

The chirp signals 308 accounts for multi-path propagation and direction-dependent delays, accurately representing mixed reflections in multi-person scenarios. In other words, the chirp signals 308 corresponds to the multiple persons.

Through this FMCW-based MIMO radar architecture, the system leverages the MIMO configuration of multiple transmit and receive antennas along with frequency-modulated chirps to achieve high-resolution estimation of human position and motion information.

Referring again to FIG. 1, after the radar transceiver 102 receives the reflected signals 110, which correspond to the chirp signals 308 in FIG. 3, the signal processing unit 104 is configured to process the reflected signals 110. As shown in FIG. 2, in step S204, the signal processing unit 104 is configured to process the reflected signals 110 to obtain signals corresponding to the multiple persons.

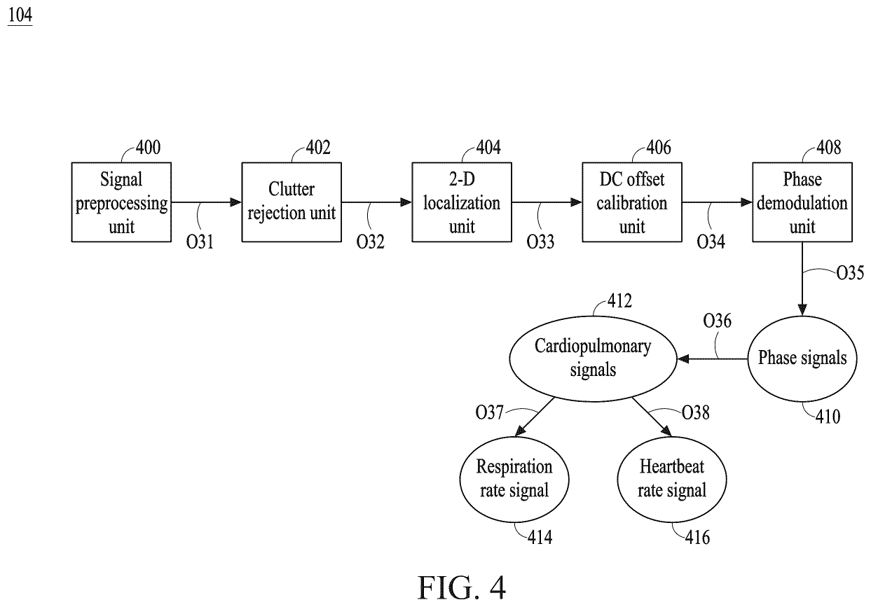

Referring to FIG. 4, FIG. 4 is a functional block diagram of a signal processing unit 104 in FIG. 1, according to an embodiment of the present disclosure. The signal processing unit 104 includes a signal preprocessing unit 400, a clutter rejection unit 402, a 2-D location unit 404, a DC offset calibration unit 406, and a phase demodulation unit 408.

In operation O31, the signal preprocessing unit 400 is configured to perform the de-ramping and in-phase/quadrature (I/Q) sampling to normalize the reflected signals 110. The normalized reflected signals of the kth analog-to digital converted (ADC) sample for the lth chirp can be expressed as:

$${x}_{m , n} ( k , l ) = {\sum }_{p = 1}^{P} {e}^{j 2 π} \{ {f}_{b , p} k {T}_{f} + λ / 2 ( {R}_{p , 0} + {R}_{p} ( {lT}_{s} ) ) + ( {u}_{n} + {u}_{m} ) / λsin ( {θ}_{p} ) \} + {δ}_{m , n} ( k {T}_{f} , {lT}_{s} ) \qquad equation ( 3 )$$

where f_{b,p}=2BRₚ(t)/cT is the beat frequency associated with the pth person 304, Rₚ(1Tₛ) is the chest displacement of the pth person 304 across slow-time due to cardiopulmonary activities, Tf is the fast-time ADC sampling interval, Ts is the slow-time chirp interval, λ is the wavelength, and δ_{m,n}(kT_{f},1Tₛ) represents the corresponding clutter and noise components.

From the above equation (3), the phase shift of the pth person 304 for the (n,m)th Tx-Rx antenna pair can be expressed as:

$${φ}_{m , n} ( l ) = {φ}_{m , n} ( {R}_{p , 0} , {θ}_{p} ) + 4 π {R}_{p} ( {lT}_{s} ) / λ \qquad equation ( 4 )$$

where φ_{m,n}(R_{p,0},θₚ)=2π/λ{2R_{p,0}+(uₙ+uₘ)sin(θₚ)} represents the phase shift induced by the relative position of the pth person 304 and the radar transceiver 102. It should be noted that φ_{m,n}(R_{p,0},θₚ) is time-invariant given that the subject of interest, i.e., the pth person 304, stays at the nominal range (radial distance) R_{p,0}, and that 4πRₚ(1Tₛ)/λ is mainly attributed to the cardiopulmonary activity of that human subject, which can be used to extract the respiration rate (RR) and heartbeat rate (HR) estimates.

In other words, φ_{m,n}(R_{p,0},θₚ) corresponds to the static phase offset induced by the subject's location and orientation, whereas 4πRₚ(1Tₛ)/λ represents the dynamic phase variations due to chest motion. Since φ_{m,n}(R_{p,0},θₚ) is time-invariant, it can be filtered out using differential methods or spatial filtering, leaving the dynamic component, i.e., 4πRₚ(1Tₛ)/λ, that carries cardiopulmonary signal signatures.

Next, the method proceeds to operation O32. In operation O32, the clutter rejection unit 402 is configured to eliminate environmental clutter from the normalized reflected signals. To generate the range profile spectrum, a K-point fast Fourier transform (FFT) is applied across the fast-time index k on x_{m,n}(k,l) as follows:

$${X}_{m , n} ( r , l ) = {FFT}_{k} \{ {x}_{m , n} ( k , l ) \} , r = 0 , … , K - 1 \qquad equation ( 5 )$$

where r denotes the range bin index.

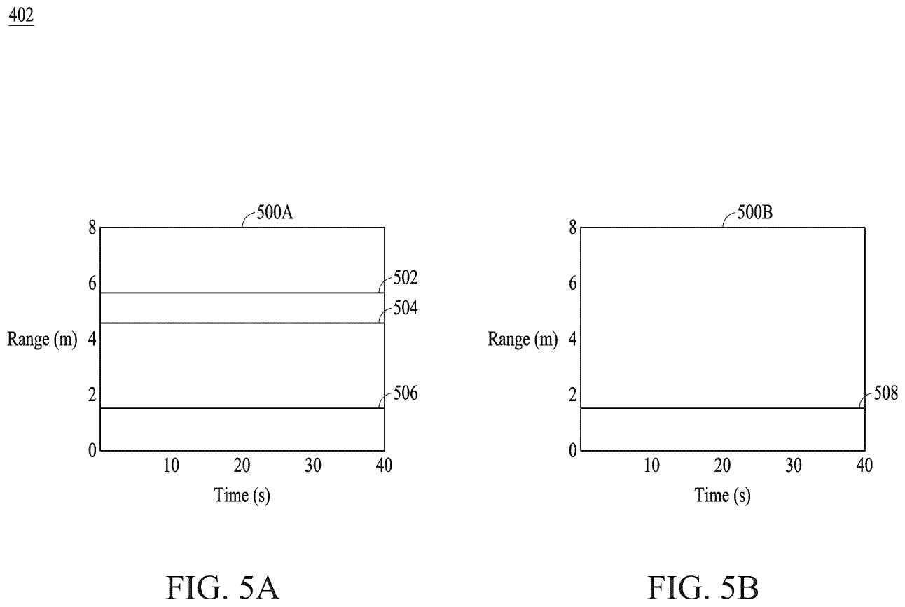

In one embodiment, there is a scenario involving two human subjects positioned at the same radial distance (e.g., approximately 1.59 m). It is observed that their signals are superimposed within the same bin along with strong environmental clutter as shown in FIG. 5A. FIG. 5A presents an exemplary range profile spectrum 500A before clutter rejection, which adversely affects the localization accuracy and the extraction of the cardiopulmonary signal. Referring to FIG. 5A, the range profile spectrum 500A before clutter rejection includes environmental clutters 502 and 504, and human subjects signals 506. To eliminate the environmental clutters 502 and 504 (which remains time-invariant across the slow time 1), a sample moving average (SMA) filter is applied, which is defined as:

$${\tilde{x}}_{m , n} ( k , l ) = {x}_{m , n} ( k , l ) - 1 / W {\sum }_{q = l - W + 1}^{l} {x}_{m , n} ( k , q ) \qquad equation ( 6 )$$

where W is the slow-time window length.

FIG. 5B presents an exemplary range profile spectrum 500B after clutter rejection. As shown in FIG. 5B, the range profile spectrum 500B after clutter rejection includes only human subjects signals 508. FIG. 5B demonstrates that, after clutter rejection, the environmental clutters 502 and 504 have been effectively suppressed, thereby facilitating downstream identification of the target range bin. In other words, the operation O32 effectively removes static background components while retaining dynamic variations associated with the cardiopulmonary signal.

Next, the method proceeds to operation O33. In operation O33, the 2-D localization unit 404 is configured to obtain range and azimuth information from the normalized reflected signals with the environmental clutter eliminated, and to determine enhanced signals based on the range and azimuth information. It should be noted that FMCW-based MIMO radar architecture shown in FIG. 3 can be formulated as an equivalent virtual array 310 having Mᵥ=MN elements by leveraging a time-division multiplexing (TDM) technique. Such an equivalent virtual array 310 can achieve higher angular resolution without the need for additional physical antennas. Therefore, the signal at the target range bin across all virtual elements is encapsulated by an Mᵥ×1 vector, expressed as:

$$x ( l ) = {[ {\tilde{X}}_{0 , 0} ( l ) , {\tilde{X}}_{0 , 1} ( l ) , … , {\tilde{X}}_{M - 1 , N - 1} ( l ) ]}^{T} \qquad equation ( 7 )$$

where \tilde{X}_{m,n}(l) denotes the range profile signal at the target range bin of the lth chirp corresponding to the (n,m)th channel pair, and (·)^{T} denotes the transpose operator.

With reference to the equation (7), an FFT is then applied across the virtual array dimension to achieve the azimuth location information of an individual. As a result, the range and azimuth location information for each individual subject is obtained. This allows the system to determine the direction of each human subject. In addition, the range and azimuth location information can be utilized to separate, and thereafter extract, accurate and reliable RR and HR estimates of an individual.

To enhance spatial resolution and suppress off-axis interference, a Capon beamformer is then used to determine the enhanced signal, expressed as:

$${y}_{p} ( l ) = {( {\hat{R}}_{x}^{- 1} a ( {\hat{θ}}_{p} ) / {a}^{H} ( {\hat{θ}}_{p} ) {\hat{R}}_{x}^{- 1} {a}^{H} ( {\hat{θ}}_{p} ) )}^{H} x ( l ) \qquad equation ( 8 )$$

where \hat{θ}ₚ is defined as the estimated direction corresponding to the pth person 304,

$${\hat{R}}_{x} = {\sum }_{l = 0}^{L - 1} x ( l ) {x}^{H} ( l )$$

denotes the covariance matrix of the virtual array output, and a (\hat{θ}ₚ) denotes the array steering vector towards the direction \hat{θ}ₚ. The equation (8) enables 2-D localization in range and azimuth, allowing downstream algorithms to isolate each human subject's signal and reliably extract individual RR and HR estimates, even in the presence of overlapping or closely spaced human targets.

Next, the method proceeds to operation O34. In operation O34, the DC offset calibration unit 406 is configured to compensate for a phase shift of the enhanced signals. To extract accurate cardiopulmonary signals 412 from individual targets, it is essential to address residual distortions in the beamformed signal caused by static clutter coupling and spectral leakage. Specifically, the enhanced signal yₚ(l) output from the Capon beamformer often exhibits inter-modulation interference and centroid drift in the complex plane due to DC offset, which can distort the phase information related to RR and HR estimation.

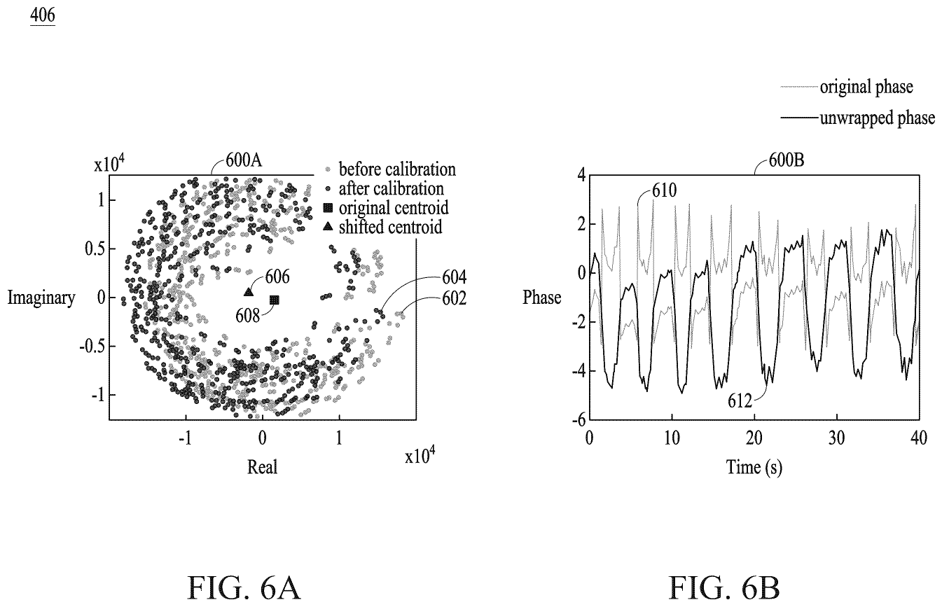

To mitigate this issue, the DC offset calibration formulated as the circle fitting problem is applied in the present disclosure. FIG. 6A is a schematic diagram illustrating the circle fitting 600A applied to the phase data before and after DC offset calibration, according to an embodiment of the present disclosure. As shown in FIG. 6A, the circle fitting 600A includes grey dots 602, black dots 604, a triangle dot 606, and a square dot 608. The grey dots 602 indicate the phase trajectory before calibration, the black dots 604 indicate the phase trajectory after calibration, the triangle dot 606 indicates the shifted centroid of the phase, and the square dot 608 indicates the original centroid of the phase.

The circle fitting problem is addressed using the least-squares method, which estimates the optimal center up of the circular trajectory. The corrected signal is then obtained by subtracting the estimated center from the enhanced signal yₚ(l), expressed as:

$${\tilde{y}}_{p} ( l ) = {y}_{p} ( l ) - {μ}_{p} \qquad equation ( 9 )$$

As a result, as shown FIG. 6A, the circular trajectory center after calibration is aligned with the origin. Specifically, the grey dots 602 are aligned with the center as the black dots 604 after calibration, and the triangle dot 606 is aligned with the center as the square dot 608 after calibration, indicating that the phase shift caused by the DC offset has been effectively compensated, thereby the calibrated center has been shifted to the origin.

Next, the method proceeds to operation O35. In operation O35, the phase demodulation unit 408 is configured to derive the phase signals 410 from the enhanced signals with the phase shift compensated. Since the phase shift of the enhanced signals has been calibrated, the phase signals 410 of the pth person 304 can be extracted as:

$${\hat{φ}}_{p} ( l ) = arctan ( R \{ {\tilde{y}}_{p} ( l ) \} / I \{ {\tilde{y}}_{p} ( l ) \} ) \qquad equation ( 10 )$$

where the symbols R{·} and I{·} denote the real and imaginary parts of a complex quantity, respectively.

Referring to FIG. 6B, FIG. 6B is a schematic diagram illustrating the extracted phase information 600B, according to an embodiment of the present disclosure. The original phase 610 corresponds to the phase signals 410 extracted right after the phase shift compensation of the enhanced signals. However, the original phase 610 is not smooth and continuous. To address this issue, phase unwrapping is applied to the original phase 610 to eliminate 2π-discontinuities caused by angular wrapping. This results in a clean and extended phase sequence suitable for downstream spectral analysis to estimate RR and HR.

As shown in FIG. 6B, an unwrapped phase 612 reveals a smooth time-domain waveform corresponding to cardiopulmonary activities, thereby providing a reliable basis for RR and HR frequency estimation.

Referring back to FIG. 2, after obtaining the phase signals 410 corresponding to the multiple persons, the signal processing unit 104 is further configured to apply wavelet transformation on the phase signals 410 using adjustable wavelet parameters to generate a respiration rate signal and a heartbeat rate signal for each of the multiple persons in step S206.

Referring back to FIG. 4. In operation O36, the signal processing unit 104 is further configured to decompose the phase signals 410 into resonance components having cardiopulmonary signals 412 with the phase signals being sparse in a wavelet domain based on the wavelet transformation. Specifically, since the cardiopulmonary signals 412 are nonstationary with both oscillatory and transient behaviors, conventional filtering techniques fall short in accurately isolating these resonance components from the phase signals 410 corrupted by the residual clutter and the random body movements (RBMs). To address this issue, the present disclosure provides a Resonance-Based Sparse Separation (RBSS) algorithm based on the Tunable Q-Factor Wavelet Transform (TQWT) for reliable reconstruction of cardiopulmonary signals 412 and accurate estimates of RR and HR.

Unlike the recovery of acoustic systems, the sparse signal recovery techniques applied to the phase signals 410, which are sparse in a wavelet domain based on the wavelet transformation, can be exploited to separate cardiopulmonary signals 412 from the phase signals 410 corrupted by the residual clutter and RBMs.

More specifically, the RBSS algorithm exploits the sparsity of the phase signals 410 in the wavelet domain, allowing for the effective separation of the respiratory and heartbeat components from the phase signals 410, which may be contaminated by the residual clutter and RBMs. The RBSS algorithm is formulated as the following convex optimization problem:

$${\hat{W}}_{p} = {arg}_{W} min {‖ {φ}_{p} - T Q W {T}^{- 1} ( W ) ‖}_{2}^{2} + {\sum }_{j = 1}^{J + 1} {λ}_{j} {‖ {w}_{j} ‖}_{1} \qquad equation ( 11 )$$

where φₚ=[\tilde{φ}ₚ(0), . . . , \tilde{φ}ₚ(L)]^{T} denotes the phase signal vector of the pth person 304, TQWT⁻¹(·) denotes the inverse TQWT, the matrix W=[w₁, . . . ,w_{J+1}] with wⱼ denotes the wavelet coefficient vector for the jth subband, J denotes the decomposition level of TQWT, and λⱼ is the regularization parameter for the jth subband.

The TQWT is governed by three key parameters, including Q factor, redundancy factor r, and decomposition level J. The Q factor evaluates the resonance quality of the phase signals 410. The redundancy factor r controls the frequency response overlap between the adjacent wavelets. The decomposition level J determines the frequency span of the adjacent wavelets. Specifically, a higher Q value corresponds to narrower bandwidth and greater resonance, making it suitable for modeling oscillatory physiological components such as the respiration rate and heartbeat rate. A higher redundancy factor r value results in greater overlap, which enhances spectral continuity and resolution in signal reconstruction. The choice of the decomposition level J directly affects the number of subbands available for signal decomposition and must be selected such that the frequency range effectively covers the respiration and heartbeat bands of interest.

In addition, the decomposition level J specifies the total number of subbands, with the maximum value given by:

$${J}_{max} = ⌊ log ( {N}_{y} / 4 ( Q + 1 ) ) / log ( ( Q + 1 ) r / ( Q + 1 ) 2 r ) ⌋ \qquad equation ( 12 )$$

where N_{y} is the length of phase signals 410, and └·┘ denotes the floor operation. It is important to note that for the phase signals 410, the precise selection of the Q factor value is crucial to achieve satisfactory decomposition under resonance conditions.

In one embodiment, since the selection of the Q factor value significantly affects the decomposition fidelity, the signal processing unit 104 is further configured to determine the optimal value of the Q factor based on maximum reconstruction energy of the cardiopulmonary signals 412. The estimate of the optimal value of the Q factor is given by:

$$\hat{Q} = {arg}_{Q} min {‖ TQW {T}^{- 1} ( {\hat{W}}_{p , v s} ) ‖}_{2}^{2} \qquad equation ( 13 )$$

where Ŵ_{p,vs} denotes wavelet coefficients with corresponding center frequencies falling within the normal RR and HR frequency range.

In operations O37 and O38, to efficiently solve the convex optimization problem with reference to the equations (12) and (13), the Split Augmented Lagrangian Shrinkage Algorithm (SALSA) can be employed to solve the equation (11). This results in a high-fidelity reconstruction of the original cardiopulmonary waveforms. The optimal wavelet basis that matches the pth person's cardiopulmonary signals 412 for each of the multiple persons can then be achieved, leading to reliable reconstruction of respiratory and heartbeat waveforms. Accordingly, the respiration rate signal 414 and heartbeat rate signal 416 can be estimated from the dominant peak frequencies within the breathing region and cardiac region spectrums.

Therefore, by jointly tuning these three parameters (i.e., the Q factor, the redundancy factor r, and the decomposition level J), the TQWT framework can be adapted to match the spectral and resonance characteristics of the phase signals 410. This enables the selection of an optimal wavelet basis for sparse reconstruction and accurate frequency estimation of the cardiopulmonary signals 412 to generate the respiration rate signal 414 and heartbeat rate signal 416.

Referring back to FIG. 2. In step S208, the biometric processing unit 106 is configured to process the heartbeat rate signal 112 of each of the multiple persons using a biometric model to determine a blood pressure (BP) signal 114 of the person.

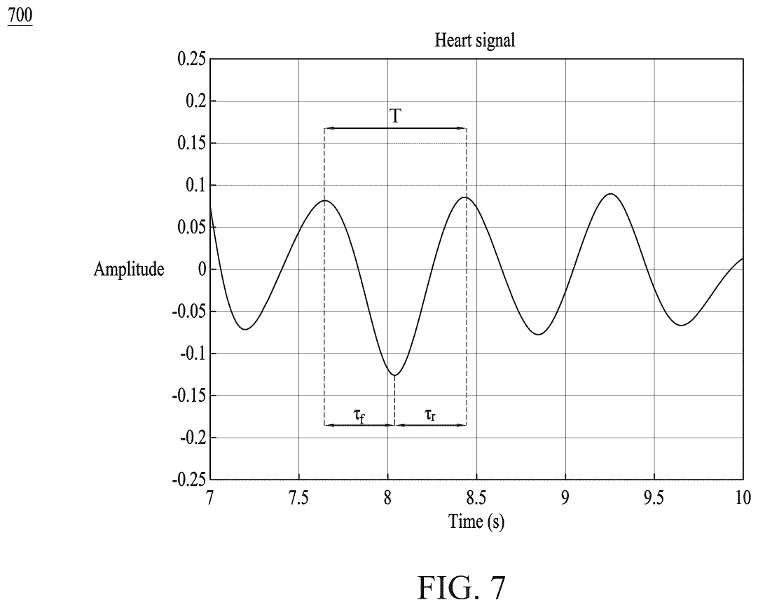

As shown in FIG. 7, FIG. 7 presents an exemplary reconstructed heartbeat waveform 700, according to an embodiment of the present disclosure. In this embodiment, the biometric model is a two-element Windkessel model. The reconstructed heartbeat waveform 700 from the heartbeat rate signal 112 illustrates the BP estimate of the pth person 304 can be achieved by using the two-element Windkessel model and the period of cardiac contraction and dilation.

More specifically, the diastolic blood pressure (DBP) and systolic blood pressure (SBP) of the pth person 304 can be estimated using the following equations:

$$D B {P}_{est} = {DBP}_{0} \cdot {e}^{- β \cdot ( {τ}_{f} - {τ}_{f 0} )} \qquad equation ( 14 )$$

where DBP₀ denotes a reference value of diastolic blood pressure obtained through multiple measurements, β denotes a decay coefficient determined via model fitting, τ_{f} denotes the cardiac contraction duration of the current beat, and τ_{f0} denotes the mean contraction time across previous beats;

$$P {P}_{est} = {PP}_{0} \cdot \frac{τ {f}^{2} / T}{τ f {0}^{2} / {T}_{0}} \qquad equation ( 15 )$$

where the PP denotes the pulse pressure, PP₀ denotes a reference value of pulse pressure obtained through multiple measurements, T denotes the time of a complete heartbeat cycle (including both systolic and diastolic phases), T₀ denotes the mean of heartbeat period.

The SBP is then given by:

$$S B {P}_{est} = D B {P}_{est} + P {P}_{est} \qquad equation ( 16 )$$

This technique enables dynamic blood pressure estimation solely based on the phase-resolved heartbeat waveform extracted from radar measurements, without the need for additional physiological sensors. It is particularly suitable for application in remote health monitoring, eldercare systems, and sleep analysis.

Referring back to FIG. 2. In step S210, the vital sign estimation unit 108 is configured to estimate, for each of the multiple persons, a respiration rate value 122, a heartbeat rate value 124, and a blood pressure value 120 based on the respiration rate signal 116, the heartbeat rate signal 118, and the blood pressure signal 114 of the person, respectively. The vital sign estimation unit 108 estimates the respiration rate value 122 based on the respiration waveform of the respiration rate signal 116, and estimates the heartbeat rate value 124 and the blood pressure value 120 based on the heartbeat waveform derived from the heartbeat rate signal 118.

The vital sign estimation unit 108 is operatively coupled to the signal processing unit 104 and the biometric processing unit 106 to receive, for each person, the respiration rate signal 116, the heartbeat rate signal 118, and the blood pressure signal 114, respectively.

Specifically, the respiration rate value 122 is derived from the respiration waveform embedded in the respiration rate signal 116. Estimation of the respiration rate value 122 by the vital sign estimation unit 108 may involve frequency domain analysis, peak detection, periodicity analysis, and/or wavelet decomposition to identify the number of respiration cycles per unit time. The heartbeat rate value 124 and the blood pressure value 120 are derived from the heartbeat waveform embedded in the heartbeat rate signal 118. The vital sign estimation unit 108 estimates the heartbeat rate value 124 by analyzing the time intervals between successive R-waves, S-waves, or other ventricular contraction indicators. The vital sign estimation unit 108 estimates the blood pressure value 120, including both systolic and diastolic components, by extracting physiological timing parameters such as the cardiac contraction time (τ_{f}) and total cardiac cycle duration (T), and applying computational models such as the two-element Windkessel model or other pulse dynamic models.

By employing the above techniques, the vital sign estimation unit 108 enables accurate, contactless, and real-time assessment of physiological metrics across multiple individuals without relying on conventional contact-based sensors (e.g., ECG electrodes or pressure cuffs), thereby offering broad applicability in smart healthcare, remote monitoring, sleep analysis, and related domains.

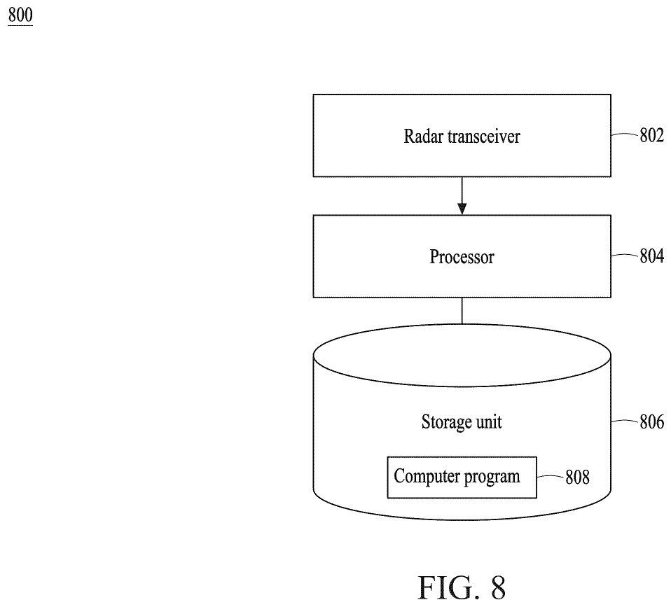

Referring to FIG. 8, FIG. 8 is a system block diagram of a vital sign monitoring system 800, according to an embodiment of the present disclosure. The vital sign monitoring system 800 includes a radar transceiver 802, a processor 804, and a storage unit 806. The radar transceiver 802 is configured to transmit radar signals toward the scene and receive reflected signals corresponding to the multiple persons. The storage unit 806 is coupled to the processor 804, and is configured to store the computer program 808.

The computer program 808 includes instructions that causes the processor 804 to perform the following operations: process the reflected signals 110 to obtain phase signals 410 corresponding to the multiple persons, and to apply wavelet transformation on the phase signals 410 using adjustable wavelet parameters to generate, for each of the multiple persons, a respiration rate signal 116 and a heartbeat rate signal 118; process the heartbeat rate signal 118 of each of the multiple persons using a biometric model 106 to determine a blood pressure signal 114 of the person; estimate, for each of the multiple persons, a respiration rate value 122, a heartbeat rate value 124, and a blood pressure value 120 based on the respiration rate signal 116, the heartbeat rate signal 118, and the blood pressure signal 114 of the person, respectively.

The vital sign monitoring system 800 may be implemented using any type of computing device with processing capabilities, such as a personal computer (e.g., a desktop computer or a laptop), a server computer, a mobile device (e.g., a tablet or a smartphone), or an embedded system, but the present disclosure is not limited thereto.

The processor 804 may include any one or more general-purpose or dedicated processors, or any combination thereof, for executing instructions. In a typical embodiment, the processor 804 may include a central processing unit (CPU) and a graphics processing unit (GPU), wherein the GPU is generally more efficient than the CPU in handling machine learning-related tasks. Accordingly, tasks may be distributed based on the characteristics of the CPU and GPU. For example, tasks such as acquiring signal data or communicating with other devices (e.g., display devices) may be assigned to the CPU, while tasks related to signal processing and/or model training may be delegated to the GPU.

The storage unit 806 may be any device including non-volatile memory, such as read-only memory (ROM), electrically erasable programmable read-only memory (EEPROM), flash memory, or non-volatile random access memory (NVRAM). Examples of such devices include, but are not limited to, hard disk drives (HDDs), solid-state drives (SSDs), or optical discs, but the present disclosure is not limited thereto.

As shown in FIG. 8, the storage unit 806 stores the computer program 808, which may be software or firmware, including a sequence or set of instructions executable by a computer system. The computer program 808 may be written in any one or more programming languages, such as Java, C, C#, C++, Python, or the like, but the present disclosure is not limited thereto. When the processor 804 loads the computer program 808 from the storage unit 806, the instructions cause the processor 804 to execute the method for detecting multiple vital signs of multiple persons in a scene as disclosed herein.

The various embodiments disclosed herein provide a system capable of simultaneous multi-person detection in a shared environment, which incorporates several key technical advantages to address real-world challenges in contactless vital sign monitoring. The system exhibits strong robustness against background clutter and random body movements, enables long-range monitoring, and achieves accurate estimation of vital signs such as blood pressure, heartbeat rate, and respiration rate at the same distance. Accordingly, the proposed system significantly broadens the practical applicability, reliability, and scalability of contactless vital sign detection technologies. It is particularly well-suited for use in smart healthcare systems, remote patient monitoring, ambient assisted living, sleep health analytics, and group health and safety surveillance, among other domains. These advancements mark a substantial step forward in enabling high-fidelity, multi-user physiological sensing in unconstrained and real-world environments

The above paragraphs are described with multiple aspects. Obviously, the teachings of the specification may be performed in multiple ways. Any specific structure or function disclosed in examples is only a representative situation. According to the teachings of the specification, it should be noted by those skilled in the art that any aspect disclosed may be performed individually, or that more than two aspects could be combined and performed.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A vital sign monitoring system for detecting multiple vital signs of multiple persons in a scene, comprising:
a radar transceiver, configured to transmit radar signals toward the scene and receive reflected signals corresponding to the multiple persons;
a signal processing unit configured to process the reflected signals to obtain phase signals corresponding to the multiple persons, and to apply wavelet transformation on the phase signals using adjustable wavelet parameters to generate, for each of the multiple persons, a respiration rate signal and a heartbeat rate signal;
a biometric processing unit, configured to process the heartbeat rate signal of each of the multiple persons using a biometric model to determine a blood pressure signal of the person; and
a vital sign estimation unit configured to estimate, for each of the multiple persons, a respiration rate value, a heartbeat rate value, and a blood pressure value based on the respiration rate signal, the heartbeat rate signal, and the blood pressure signal of the person, respectively.

2. The vital sign monitoring system as claimed in claim 1, wherein the signal processing unit further comprises:
a 2-D localization unit, configured to apply a Fast Fourier Transform to the reflected signals to obtain range and azimuth information, and apply a beamformer based on the range and azimuth information to determine enhanced signals with respect to each of the multiple persons;
a DC offset calibration unit, configured to compensate for a phase shift of the enhanced signals; and
a phase demodulation unit, configured to derive the phase signals from the enhanced signals with the phase shift compensated.

3. The vital sign monitoring system as claimed in claim 2, wherein the signal processing unit comprises:
a signal preprocessing unit, configured to normalize the reflected signals; and
a clutter rejection unit, configured to eliminate environmental clutter from the normalized reflected signals;
wherein the 2-D localization unit receives the normalized reflected signals with environmental clutter eliminated from the clutter rejection unit.

4. The vital sign monitoring system as claimed in claim 1, wherein the signal processing unit is further configured to decompose the phase signals into resonance components having cardiopulmonary signals with the phase signals being sparse in a wavelet domain based on the wavelet transformation.

5. The vital sign monitoring system as claimed in claim 4, wherein the signal processing unit is further configured to, for each of the multiple persons, determine an optimal wavelet basis based on an optimal value of the adjustable wavelet parameters that matches the cardiopulmonary signals, and reconstructs a respiration waveform and a heartbeat waveform from the cardiopulmonary signals based on the optimal wavelet basis.

6. The vital sign monitoring system as claimed in claim 5, wherein the adjustable wavelet parameters comprise a Q factor, a redundancy factor, and a decomposition level.

7. The vital sign monitoring system as claimed in claim 6, wherein the Q factor evaluates resonance quality of the phase signals, and the redundancy factor controls a frequency response overlap between adjacent wavelets, and the decomposition level determines a frequency span of the adjacent wavelets.

8. The vital sign monitoring system as claimed in claim 6, wherein the signal processing unit is further configured to determine the optimal value of the Q factor based on maximum reconstruction energy of the cardiopulmonary signals.

9. The vital sign monitoring system as claimed in claim 8, wherein the respiration waveform and the heartbeat waveform are reconstructed based on the optimal value of the Q factor with corresponding center frequencies falling within a respiration waveform frequency range and a heartbeat waveform frequency range, respectively.

10. The vital sign monitoring system as claimed in claim 9, wherein the biometric processing unit determines the blood pressure signal based on the heartbeat waveform using the biometric model, and
wherein the vital sign estimation unit estimates the respiration rate value based on the respiration waveform, and the heartbeat rate value and the blood pressure value based on the heartbeat waveform.

11. The vital sign monitoring system as claimed in claim 1, wherein the biometric model is a two-element Windkessel model.

12. A method for detecting multiple vital signs of multiple persons in a scene, comprising:
transmitting radar signals toward the scene and receiving reflected signals corresponding to the multiple persons by a radar transceiver; and
by a processor, executing steps comprising:
processing the reflected signals to obtain phase signals corresponding to the multiple persons;
applying wavelet transformation on the phase signals using adjustable wavelet parameters to generate, for each of the multiple persons, a respiration rate signal and a heartbeat rate signal;
processing the heartbeat rate signal of each of the multiple persons using a biometric model to generate a blood pressure signal of the person; and
estimating, for each of the multiple persons, a respiration rate value, a heartbeat rate value, and a blood pressure value based on the respiration rate signal, the heartbeat rate signal, and the blood pressure signal of the person, respectively.

13. The method as claimed in claim 12, wherein the step of processing the reflected signals to obtain phase signals corresponding to the multiple persons further comprises:
applying a Fast Fourier Transform to the reflected signals to obtain range and azimuth information;
applying a beamformer based on the range and azimuth information to determine enhanced signals with respect to each of the multiple persons;
compensating for a phase shift of the enhanced signals; and
deriving the phase signals from the enhanced signals with the phase shift compensated.

14. The method as claimed in claim 13, wherein the step of processing the reflected signals to obtain phase signals corresponding to the multiple persons comprises:
normalizing the reflected signals; and
eliminating environmental clutter from the normalized reflected signals;
wherein the step of eliminating the environmental clutter is performed before the step of applying the Fast Fourier Transform to obtain the range and azimuth information.

15. The method as claimed in claim 12, wherein the step of processing the reflected signals to obtain phase signals corresponding to the multiple persons further comprises:
decomposing the phase signals into resonance components having cardiopulmonary signals with the phase signals being sparse in a wavelet domain based on the wavelet transformation.

16. The method as claimed in claim 15, wherein the step of applying wavelet transformation on the phase signals using adjustable wavelet parameters to generate, for each of the multiple persons, a respiration rate signal and a heartbeat rate signal comprises:
determining an optimal wavelet basis based on an optimal value of the adjustable wavelet parameters that matches the cardiopulmonary signals for each of the multiple persons; and
reconstructing a respiration waveform and a heartbeat waveform from the cardiopulmonary signals based on the optimal wavelet basis.

17. The method as claimed in claim 16, wherein the adjustable wavelet parameters comprise a Q factor, a redundancy factor, and a decomposition level.

18. The method as claimed in claim 17, wherein the Q factor evaluates resonance quality of the phase signals, and the redundancy factor controls a frequency response overlap between adjacent wavelets, and the decomposition level determines a frequency span of the adjacent wavelets.

19. The method as claimed in claim 17, wherein the step of applying the wavelet transformation on the phase signals using adjustable wavelet parameters to generate, for each of the multiple persons, the respiration rate signal and the heartbeat rate signal further comprises:
determining the optimal value of the Q factor based on maximum reconstruction energy of the cardiopulmonary signals.

20. The method as claimed in claim 19, wherein the respiration waveform and the heartbeat waveform are reconstructed based on the optimal value of the Q factor with corresponding center frequencies falling within a respiration waveform frequency range and a heartbeat waveform frequency range, respectively.

21. The method as claimed in claim 20, wherein:
the step of processing the heartbeat rate signal of each of the multiple persons using the biometric model to determine the blood pressure signal of the person comprises:
determining the blood pressure value based on the heartbeat waveform using the biometric model;
the step of estimating, for each of the multiple persons, the respiration rate value, the heartbeat rate value, and the blood pressure value based on the respiration rate signal, the heartbeat rate signal, and the blood pressure signal of the person comprises:
estimating the respiration rate value and the heartbeat rate value based on the respiration waveform and the heartbeat waveform, respectively.

22. A vital sign monitoring system for detecting multiple vital signs of multiple persons in a scene, comprising:
a radar transceiver, configured to transmit radar signals toward the scene and receive reflected signals corresponding to the multiple persons;
a processor; and
a storage unit, coupled to the processor, and configured to store a computer program comprising instructions that, when executed by the processor, causes the processor to:
process the reflected signals to obtain phase signals corresponding to the multiple persons, and to apply wavelet transformation on the phase signals using adjustable wavelet parameters to generate, for each of the multiple persons, a respiration rate signal and a heartbeat rate signal;
process the heartbeat rate signal of each of the multiple persons using a biometric model to determine a blood pressure signal of the person; and
estimate, for each of the multiple persons, a respiration rate value, a heartbeat rate value, and a blood pressure value based on the respiration rate signal, the heartbeat rate signal, and the blood pressure signal of the person, respectively.

23. The vital sign monitoring system as claimed in claim 22, wherein:
the phase signals are decomposed into resonance components having cardiopulmonary signals with the phase signals being sparse in a wavelet domain based on the wavelet transformation;
the adjustable wavelet parameters comprise a Q factor, a redundancy factor, and a decomposition level; and
the Q factor evaluates resonance quality of the phase signals, and the redundancy factor controls a frequency response overlap between adjacent wavelets, and the decomposition level determines a frequency span of the adjacent wavelets.
